(19) United States
(12) Patent Application Publication
(10) Pub. No.: US 2022/0265159 A1
(43) Pub. Date: Aug. 25, 2022

(54) Wearable Low Power Continuous Perinatal Monitor

(71) Applicant: Javelin Devices Inc., Austin, TX (US)

(72) Inventors: Lucas Martin Holt, Austin, TX (US); Andrew Peter Wang, Austin, TX (US); Evan B. Bartilson, Millcreek, UT (US); Benjamin Michael Bright, Austin, TX (US)

(21) Appl. No.: 17/652,633

(22) Filed: Feb. 25, 2022

Related U.S. Application Data
(60) Provisional application No. 63/153,748, filed on Feb. 25, 2021.

(51) Int. Cl. A61B 5/024; A61B 5/02; A61B 5/11; A61B 5/00; A61B 5/256; A61B 5/257; A61B 7/00

(52) U.S. Cl. CPC A61B 5/02411; A61B 5/02028; A61B 5/11; A61B 5/7207; A61B 5/0004; A61B 5/7475; A61B 5/256; A61B 5/257; A61B 5/6804; A61B 7/006; A61B 2562/0219; A61B 2562/166; A61B 2560/0209

(57) ABSTRACT

A sensing device for sensing perinatal maternal uterine activity and fetal heart activity is provided. The sensing device includes a body with an electromechanical system and a housing, a passive acoustic system with a plurality of microphones and an acoustic waveguide, an attachment component, an accelerometer, a signal analysis system including a microcontroller unit, and a wireless transceiver. The signal analysis system is configured to process biopotential signals and acoustic signals detected by the passive acoustic system and to reduce motion artifacts from the signals using the accelerometer.

### RELATED APPLICATIONS

This application claims the benefit of U.S. Provisional Patent Application No. 63/153,748, filed Feb., 25, 2021, which is incorporated by reference herein.

### TECHNICAL FIELD

A system and method for monitoring human heart activity is disclosed. Specifically, a perinatal device that simultaneously senses maternal uterine activity and fetal heart activity during pregnancy is disclosed.

### BACKGROUND

Perinatal monitoring is a tool for healthcare professionals and mothers who want to ensure the health and safety of babies during pregnancy, particularly labor and delivery, as well as the comfort of the mother during these events. These devices determine the health status of the fetus by measuring fetal heart rate (FHR) as well as maternal contractions during pregnancy leading up to the delivery.

There are various known systems that measure fetal heart hate. For example, cardiotocography (CTG) is used for determining fetal conditions but relies on an apparatus which includes a doppler-based ultrasound transducer to detect FHR and a tocodynamometer pressure transducer for sensing maternal contractions. CTGs are typically encased in a waterproof housing and placed on the mother using a belt. CTGs allow for continuous monitoring by a professional healthcare provider.

These devices are not robust enough to provide accurate data while maintaining the comfort of pregnant patients. For example, during labor, the mother will move around in the hospital bed or ambulate which causes the sensors to move such that the signals of interest may no longer be reliably detected. These types of sensors are sensitive to placement error and require a healthcare professional to perform sensor placement, which prevents independent or in-home use by the pregnant user.

CTGs suffer further limitations. They are not wireless due to the power requirements of ultrasound. When the mother is ambulating or showering, the monitor and cables remain a cumbersome attachment.

A further limitation is that ultrasound uses high frequency acoustic energy making it difficult to detect the FHR in high body mass index (BMI) patients because the high-frequency sound energy dissipates in the adipose tissue.

The need exists for an apparatus that can monitor FHR and maternal contractions in a clinical setting while enabling the mother to maneuver independently. A further need exists for a sensing method that is robust to the variability in maternal BMI.

Doppler ultrasound solutions like the doppler fetal monitor have managed to reduce power requirements and enable portable monitoring. However, ultrasound remains invasive in nature, directing high frequency vibrations toward the developing fetus's heart valve. Recent studies and guidance indicate excessive ultrasound measurements transmit active energy waves into the fetus for an extended duration and can cause heating and cavitation of tissue. These effects are not well understood, but still result in some mothers' preference to avoid the additional health risks of prolonged ultrasound exposure. There exists a need for a safe alternative to ultrasound to enable continuous monitoring of FHR.

The known fetal electrocardiogram (fECG) solutions available use electrocardiography and electrohysterography (EHG) to detect FHR and maternal contractions. fECGs comprise multiple electrodes attached to the mother's abdomen using adhesive. Due to the lower power requirements of this method, the monitors are wireless. The fECG method has success with high BMI mothers because it senses electrical activity with similar quality despite variance in adipose tissue. fECG is non-invasive and doesn't direct energy towards the fetal tissues.

However, fECG monitors depend on precise placement of adhesive based electrodes. The precision of placement of electrodes is critical for accurate sensing of the FHR as well as maintaining secure contact throughout the labor process despite movement of the mother and the fetus. Due to the adhesive, if the location is not correct then the electrodes need to be removed and a new set of electrodes need to be placed. Also, if the fetus moves inside of the uterus to a location that is not suitable for the current location of the electrodes, then the electrode placement process will have to be redone. The precision required for electrode placement for current fECG monitoring systems presents a challenge for mothers attempting to perform it independently. The sensitivity to electrode placement and fetus orientation prevents the monitoring apparatus from being robust enough for continuous use outside a clinical environment.

The available perinatal monitors carry a high cost that makes them largely unavailable to certain socioeconomic groups. Based on these limitations, there exists a need for a cost-effective device to monitor FHR as well as maternal contractions that is not cumbersome to place on the patient and is able to maintain accurate measurements throughout the laboring process in environments beyond the clinic. There further exists a need for a methodology to reduce the challenge of sensor placement and the detriment of sensor misplacement to enable independent application and monitoring by the mother.

### SUMMARY

A sensing device for sensing perinatal maternal uterine activity and fetal heart activity is provided. The sensing device includes a body with an electromechanical system and a housing, a passive acoustic system with a plurality of microphones and an acoustic waveguide, an attachment component, an accelerometer, a signal analysis system including a microcontroller unit, and a wireless transceiver. The signal analysis system is designed to process biopotential signals and acoustic signals detected by the passive acoustic system. The signal analysis system is also designed to reduce motion artifacts from the signals using the accelerometer.

In another instance, a method of determining a signal quality index of acoustic fetal heart sounds is provided. The method includes providing an electromechanical system for measuring fetal heart activity using a passive acoustic system, processing the fetal heart activity using a signal analysis system, generating a fetal heart signal from the measured fetal heart activity, and calculating the acoustic fetal heart signal quality index according to the input of the signal analysis system. The method also includes processing new signals as a pressure and a position of the system is updated. A user interface is also used to provide instructions to the user on how to adjust the pressure and position of the system. The user interface can be configured to update instructions and display updated instructions based on user input and calculated measurements.

In another embodiment, a method for labeling clinically significant events based on recorded cardiac activity is provided. The method includes providing a visual display with data associated with fetal heart activity and maternal uterine activity, receiving an input from a user, associating the input with either a start or a stop time (or a combination thereof) of the heart activity considered a clinically significant event, providing pre-populated labels for the user to select in order to label the clinically significant event, and prompting the user to review and finalize the inputs associated with the labeled activity.

### BRIEF DESCRIPTION OF THE DRAWINGS

In the drawings, closely related figures have the same number but different alphabetic suffixes. Some embodiments of the present invention are displayed below as an example and are not limited by the figures of the accompanying drawings in which:

FIG. 1A is a top isometric view of a sensing device according to one embodiment;

FIG. 1B is a bottom isometric view of the sensing device of FIG. 1A;

FIG. 2 is an exploded view of a body of the sensing device of FIGS. 1A and 1B;

FIG. 3A is a top plan view of the sensing device of FIG. 2;

FIG. 3B is a cross-sectional view of the sensing device of FIG. 3A, taken along line 41 in FIG. 3A;

FIG. 4A is a front plan view of the sensing device described herein that is attached to a maternal abdomen using a garment and externally placed adhesive electrodes according to one embodiment;

FIG. 4B is a side elevational view of the sensing device described herein attached to a maternal abdomen using a garment and externally placed adhesive electrodes according to one embodiment;

FIG. 5 is an isometric view of an adhesive patch interface with integrated electrodes attached to the sensing device according to one embodiment;

FIG. 6A is a top plan view of the sensing device according to one embodiment;

FIG. 6B is a cross-section view of the sensing device of FIG. 6A, taken along line 42 in FIG. 6A, with a screw-type adjustable pressure mechanism in a retracted state according to one embodiment;

FIG. 6C is a cross-section view of the sensing device of FIG. 6A, taken along line 42 in FIG. 6A, with a screw-type adjustable pressure mechanism in an extended state according to one embodiment;

FIG. 6D is a cross-section view of the sensing device of FIG. 3A, taken along line 42 in FIG. 6A, with a spring-type adjustable pressure mechanism in a retracted state according to one embodiment;

FIG. 6E is a cross-section view of the sensing device of FIG. 3A, taken along line 42 in FIG. 6A, with a spring-type adjustable pressure mechanism in an extended state according to one embodiment;

FIG. 7A is a side elevational view of the sensing device of FIG. 6A with a pressure level indicator in a retracted state according to one embodiment;

FIG. 7B is a side elevational view of the sensing device of FIG. 6A with a pressure level indicator in a retracted state according to one embodiment;

FIG. 8 is a graph depicting high quality acoustic fetal heart signal in both raw form, and processed utilizing the sensing device described herein;

FIG. 9 is a graph depicting poor quality acoustic fetal heart signal in both raw and processed form;

FIG. 10A is a front elevational view of a maternal abdomen that has been divided into quadrants that may be used for determining the recommended position for measuring acoustic fetal heart activity according to one embodiment;

FIG. 10B shows a maternal abdomen that has been further subdivided into smaller quadrants that may be used for determining the recommended position for measuring acoustic fetal heart activity according to one embodiment;

FIG. 11 is a flow diagram of a method to implement labeling of clinically significant events according to one embodiment;

FIG. 12A is an example of a healthcare provider interface for manual label entry according to one embodiment;

FIG. 12B is an example of a healthcare provider interface for manual label entry according to one embodiment;

FIG. 13 is a block diagram of a microphone printed circuit board used in the sensing device described herein; and

FIG. 14 is a block diagram of the main control printed circuit board used in the sensing device described herein.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Specifically, while many of the embodiments discuss a sensor device for measuring uterine activity electrohysterography, it will be appreciated by those skilled in the art that the device can be modified or otherwise adapted for other sensing applications of electromyography. Similarly, where embodiments discuss the sensor device in relation to a maternal abdomen, it will be appreciated that the embodiment can be modified or adapted to interface with other areas of a body, not just the maternal abdomen.

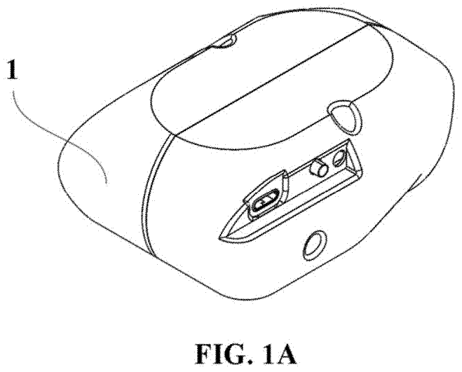

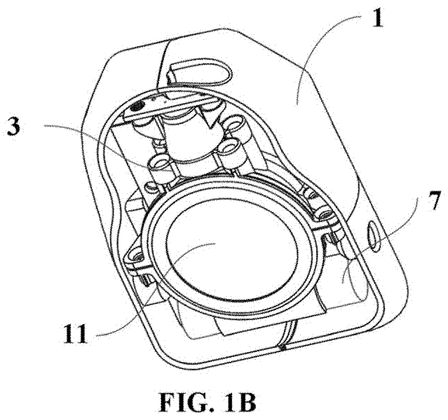

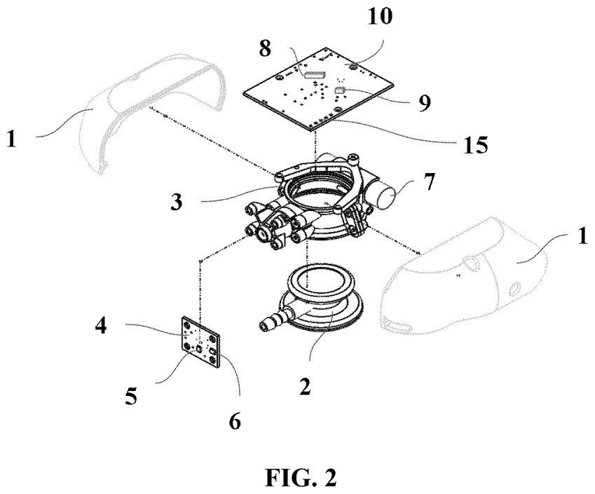

FIGS. 1A-1B illustrate a sensing device according to one embodiment. The sensing device can be used to sense maternal uterine activity and fetal heart activity during any gestational period of pregnancy. The sensing device can be used to sense fetal heart activity and maternal uterine activity using fECG, EHG, and acoustics. The sensing device is designed to provide a passive low power sensing system, meaning the sensing components operate without sending energy, including acoustic energy, into the fetus. The sensing device includes a body provided in the form of a housing 1 that contains the electromechanical system of the sensing device, shown in FIG. 2.

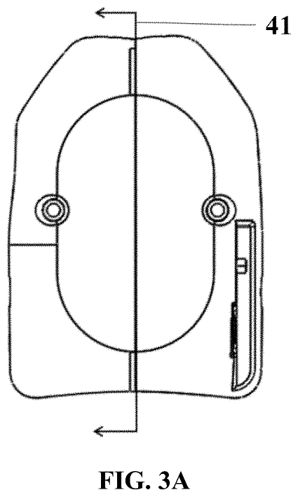

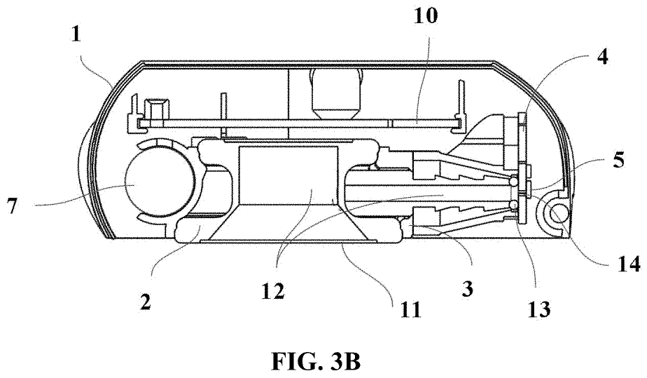

FIG. 2 shows an exploded view of one embodiment of the sensing device, including the electromechanical system with a plurality of electromechanical components disposed within the housing 1. The electromechanical system includes at least: a microphone printed circuit board (PCB) 4, an acoustic waveguide 2, an acoustic waveguide housing 3, a battery 7, an electrode connector port 15, and an accelerometer 9. The microphone PCB 4 includes a sensing microphone 5 and a noise-canceling microphone 6. Alternate embodiments may include a plurality of sensing microphones. The sensing microphone 5 is designed for transducing pressure waves produced from the maternal abdomen into electrical signals. The sensing microphone 5 in some embodiments is a pulse density modulation (PDM) microphone designed with a bottom port to allow the sensing microphone 5 to sit flat against the microphone PCB 4. In some embodiments a sealing component 13 (shown in FIG. 3B) can be used for acoustically isolating the bottom port to improve signal quality. The sealing component 13 can be used to seal the bottom port within a resonance chamber 12 (shown in FIG. 3B). The sealing component 13 can be designed to maintain a sealed pressure path from the frequency diaphragm 11 to the sensing microphone 5. The noise-canceling microphone 6 can be used for capturing external noise not associated with the maternal abdomen. In some embodiments the noise-canceling microphone 6 can be a PDM microphone designed with a bottom port to allow the noise-canceling microphone 6 to sit flush against the microphone PCB 4. In some embodiments the noise-canceling microphone 6 may interface with a sealing component to sit flat against the microphone PCB 4.

Still referring to FIG. 2, the acoustic waveguide housing 3 can be positioned such that the acoustic waveguide 2 fits within the acoustic waveguide housing 3. In some embodiments, the acoustic waveguide housing 3 can be designed to couple to the acoustic waveguide 2. The acoustic waveguide 2 can include a circular frequency diaphragm 11 (shown in FIG. 1B), and the resonance chamber 12 (shown in FIG. 3B). In some embodiments, the acoustic waveguide 2 can be provided in the form of a head of a Littman brand stethoscope, wherein the head includes the frequency diaphragm 11 and the resonance chamber 12. The frequency diaphragm 11 can be frequency tunable in response to pressure. In one embodiment, an increase in pressure will result in a higher frequency output, whereas a lower pressure value will result in a lower frequency targeted by the sensing device. Other types of acoustic waveguides may also be used such as commercially available stethoscope heads or an acoustic waveguide where the resonance chamber and the frequency diaphragm are designed to target fetal heart sounds.

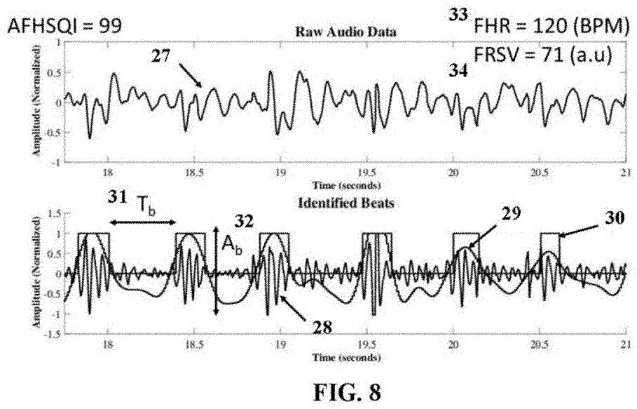

The accelerometer 9 is designed to sense motion activity from the maternal abdomen. In some embodiments, the motion activity detected from the accelerometer 9 is processed using a main control PCB 10, where the main control PCB 10 includes a microcontroller unit (MCU) 8. In some embodiments the accelerometer 9 is a commercially available ultra-low power unit with a 16-bit output with selectable full scale from a range of +−2 g to +−16 g. The MCU 8 is designed to process signals, including at least acoustic and biopotential signals. Examples of biopotential signals include, but are not limited to, electrocardiography and electromyography signals. The main control PCB 10 and the MCU 8 can be designed to process the acoustic and biopotential signals, for example, into fetal heart activity and maternal uterine activity as well. The main control PCB 10 and the MCU 8 can also be designed to reduce motion artifacts from the sensed motion activity from the accelerometer 9 to reduce interference with the signal quality of the acoustic and biopotential signals. Examples of motion artifacts include corruptions to the acoustic or biopotential signals, often a result of movement of the sensor device or pressure fluctuations between the frequency diaphragm 11 and the maternal abdomen. The sensing device is designed to provide a cardiac output using the fetal hemodynamics. Furthermore, FHR and fetal hemodynamics are extracted using digital signal processing techniques that calculate time between and amplitude of acoustic energy pulses that are associated with fetal heart beats, specifically with heart valves opening and closing. As the force of contraction, or contractility, increases, the valves close and open more forcefully causing the amplitude of the heart sound to increase. As contractility decreases, the valves will close and open less forcefully causing a decrease in amplitude of the heart sound. As contractility increases, the end-diastolic volume decreases, resulting in a greater stroke volume and thus greater cardiac output. The amplitude of the heart sounds can be calculated as described in the embodiment shown in FIG. 8 and detailed in the Fetal Heart Rate and Fetal Hemodynamics Signal Processing section below.

With further reference to the sensing device of FIGS. 1A-3B, the sensing device can be designed to detect and process audio amplitude derived relative stroke volume and cardiac output. During an acute hypoxic state, cardiac output increases as the body compensates for oxygen demand and myocardial contractility decreases in severe hypoxic states. Detecting these physiological trends along with FHR and maternal contractions can help healthcare professionals make better informed decisions on interpretation and management for fetal distress.

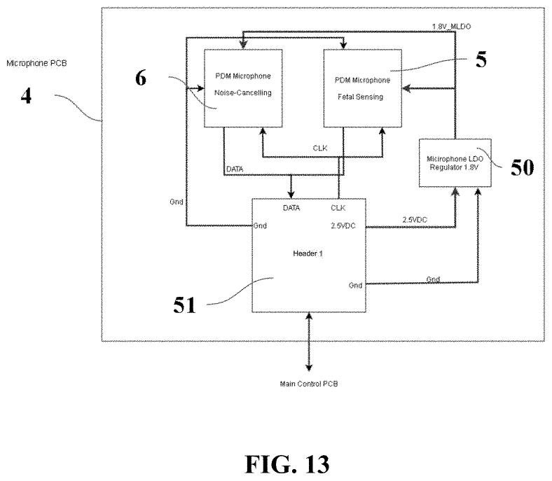

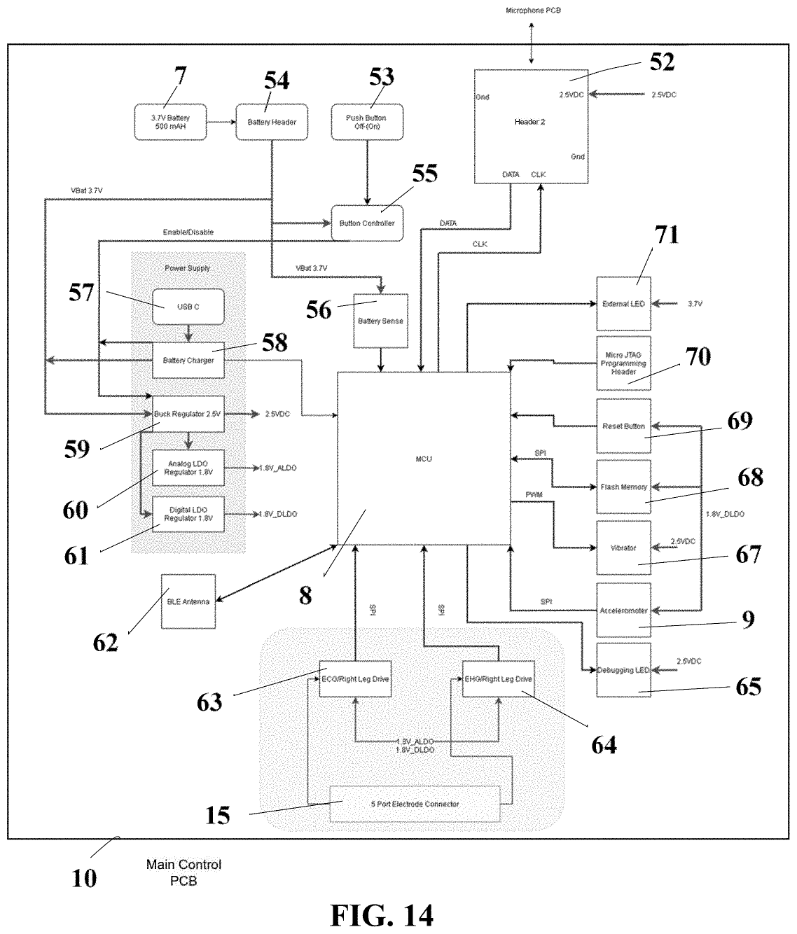

FIGS. 13 and 14 show a block diagram of the microphone PCB 4, and the main control PCB 10, respectively, according to some embodiments. The microphone PCB 4 and main control PCB 10 are connected using first header 51 and second header 52 and a cable (not shown). A voltage from a 2.5V buck regulator 59 from the main control PCB 10 is fed into a microphone linear dropout regulator (LDO) 50 and converted into 1.8V, which is then used to power the sensing microphone 5 and the noise-canceling microphone 6. Both the sensing microphone 5 and the noise-canceling microphone 6 can communicate with the MCU 8 using pulse density modulation (PDM), which has a clock and data line.

Referring to FIG. 14, the main control PCB 10 is powered by a battery 7 that is connected to the main control PCB 10 using a battery header 54 and then converted into 2.5V using the buck regulator 59. In some embodiments, the battery 7 is a rechargeable 3.7V lithium ion 500 mAH battery. The battery 7 is charged using a USB C connector 57 and a battery charging chip 58. A battery level of the battery 7 is sensed by a battery sensing circuit 56 that can send the voltage level to the MCU 8. Two LDO's are used to convert the 2.5V to 1.8V to power the analog (analog LDO 60) and digital (digital LDO 61) components on the board.

The electrodes used for fECG and EHG can be connected using a 5-port electrode connector 15 using wires. The biopotentials sensed by the electrodes feed into the fECG and EHG chips (63, 64) that communicate with the MCU 8 using Serial Peripheral Interface (SPI). The accelerometer 9 and flash memory chip 68 also communicate with the MCU 8 using SPI.

Other peripherals on the main control PCB 10 can include an external LED 71 that can be seen outside of the housing 1 of the device, a debugging LED 65, and a vibration motor 67 for haptic feedback to the user. These three peripherals can be controlled by the MCU 8 using pulse width modulation (PWM).

A reset button 69 is used to reset the MCU 8, and the programming header 70 is used to program the MCU 8 using a cable (not shown). An external push button 53 and a button controller 55 are used to turn the main control PCB 10 on and off through the buck regulator 59. BLE wireless communication may be used with a BLE antenna 62 that is connected to the transceiver on the MCU 8.

In some embodiments, the sensing device includes a transceiver on the MCU 8 designed to share or otherwise transmit information between the signal analysis system and a base station (not shown). The transceiver can be wireless in order to provide additional mobility, including the ability of the user to wear the sensing device while ambulatory, for example. The base station may include a user interface device, wherein the user interface device may be a mobile device, mobile phone, tablet, computer, or similar. The wireless capability, portability, and ability for continuous operation allow the sensing device to be used in both clinical, non-clinical, and in-home settings.

Shown in FIG. 3B, the frequency diaphragm 11 protrudes outwardly beyond the surface of the housing 1 of the device to contact the skin of the maternal abdomen and transduce vibrations from the skin into a plurality of pressure waves that move through the resonance chamber 12 and the microphone PCB 4 port hole 14 to the sensing microphone 5 that receives the pressure waves. The sensing microphone 5 transduces the plurality of pressure waves into electrical signals that can be processed by the MCU 8. A sealing component 13 is positioned between an output of the resonance chamber 12 and microphone PCB 4 port hole 14 to maintain a sealed sound path from the frequency diaphragm 11 to the sensing microphone 5 to increase pressure wave transmission. In some embodiments, the sealing component 13 can be provided in the form of a compliant sealing component. In one embodiment, the sealing component 13 is a rubber O-ring.

In one embodiment, the microphone PCB 4 and main control PCB 10 are separated with a split ground plane and a split power plane. In other embodiments, the microphone PCB 4 and the main control PCB 10 may be designed without either or both of the split ground plane and the split power plane. The microphone PCB 4 and the main control PCB may be designed with at least one trace designed to connect either or both of the split ground plane or the split power plane. Additional embodiments can include a solid ground plane and a solid power plane. The components on the microphone PCB 4 are minimized to what is necessary to power the sensing microphone 5 and the noise-canceling microphone 6. Minimizing the number of components on the microphone PCB 4 as well as the split ground and power planes decreases unwanted electrical noise and increases the transduced electrical signal quality sensed from the maternal abdomen. The sensing device is designed to operate at very low power and long runtime. In some embodiments, the sensing device is powered using a 500 mAh battery 7 (shown in FIG. 2). Some embodiments can include a wired power source or alternate battery sources to provide variability in both power consumption and runtime.

The sensing microphone 5 targets audio sensed from the maternal abdomen, but unwanted external noise can still be present. The noise-canceling microphone 6 removes external noise by capturing the unwanted external noise audio and the MCU 8 subtracts the audio from the noise-canceling microphone 6 from audio from the sensing microphone 5 to increase the signal to noise ratio of the audio sensed from the maternal abdomen. In one embodiment, the signal from the noise canceling microphone 6 is transformed into the Fourier domain using a fast Fourier transform (FFT) by the MCU 8. The MCU 8 identifies the frequencies with the highest energy. Those identified frequencies are then filtered out of the signal from the sensing microphone 5 using a band stop digital filter with cutoff thresholds set based on the high energy noise frequencies.

With further reference to FIG. 3B, the microphone PCB 4 is attached to the acoustic waveguide housing 3. The acoustic waveguide housing 3 is attached to the housing 1 of the sensing device with a fastener (not shown) for acoustic isolation of the electromechanical components and the housing 1 of the sensing device. The acoustic isolation will reduce unwanted vibrations applied to the housing 1 of the sensing device from reaching the sensing microphone 5. In some embodiments, the fastener may include a minimal screw contact or other minimal material contact designed to provide acoustic isolation.

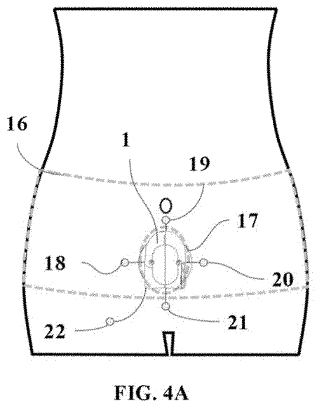

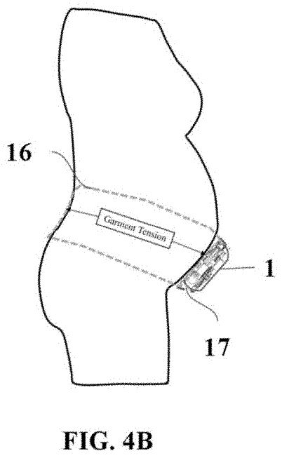

FIG. 4A shows one embodiment of the sensing device as worn on a human body. In accordance with one embodiment, a garment 16 can be worn around the maternal abdomen and the sensing device can be placed inside a garment pocket with a hole that exposes the frequency diaphragm 11 to the skin. In some embodiments, the garment 16 is designed to securely hold the sensing device against the human body. The garment 16 may be a pregnancy band, or similar stretchy, flexible, or elastic-type material strip. The garment 16 may be fastened or otherwise secured around the body in many configurations. For example, the garment 16 may be secured using a Velcro, snapfits, buttons, adhesive, hooks, or some other type of fastener of plurality of fasteners. In some embodiments there are four electrodes (18, 19, 20, 21) and a common mode drive electrode 22 integrated with the attachment component. In some embodiments, the four electrodes 18-21 and the common drive electrode 22 are individual adhesive patch electrodes that may be connected to an electrode connector port 15 in the sensing device. The four electrodes 18-21 and the common drive electrode 22 can be commercially available silver/silver chloride adhesive patch electrodes designed for ECG, EMG, fECG, or EHG measurements, or any combination thereof. The four electrodes 18-21 can be used to measure fECG and EHG from the maternal abdomen. The common mode drive electrode 22 is designed to actively cancel the common mode signal interference from the maternal body using feedback.

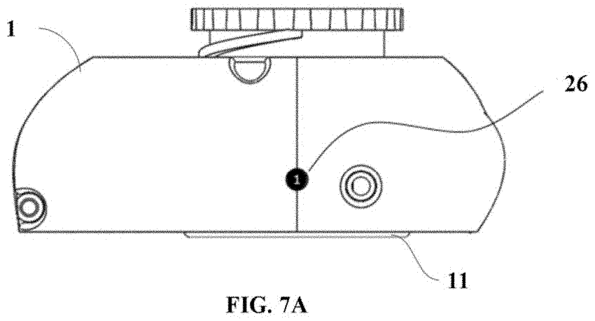

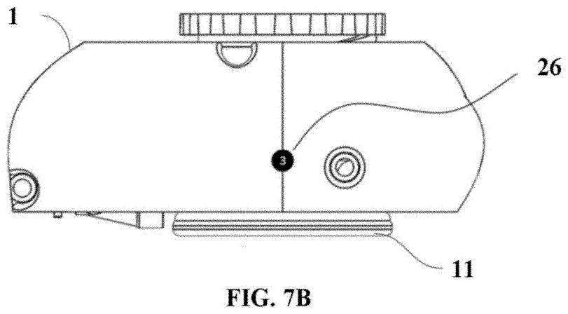

In some embodiments, a tension parameter of the garment 16 can be adjusted, as shown in FIG. 4B, resulting in changes in pressure of the frequency diaphragm 11 against the maternal abdomen. The tunable frequency diaphragm 11 targets lower frequency skin vibrations with lower pressure against the maternal abdomen and higher frequency skin vibrations with higher pressure against the maternal abdomen to improve signal quality. In one embodiment, the pressure level is associated with how far the frequency diaphragm 11 is retracted or extended as compared to a distance of the frequency diaphragm 11 to the housing 1 of the sensing device. This is shown by the indicator in FIGS. 7A and 7B by a pressure setting level “3”, for example, shown on the pressure level indicator 26 of the sensing device, the pressure setting level associated with a distance of the extension or retraction of the frequency diaphragm 11. A recommended pressure setting level can be provided or selected based on the calculated acoustic fetal heart signal quality index (AFHSQI) values.

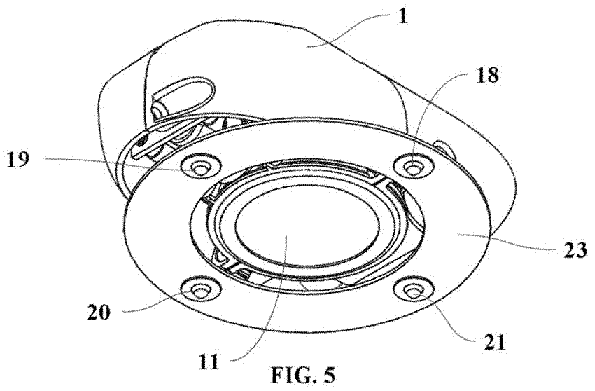

In an alternative embodiment, the housing 1 of the sensing device is attached to an adhesive patch 23 interface using snap-fit assemblies, as shown in FIG. 5. Each snap-fit assembly is associated with the four electrodes 18-21 integrated into the adhesive patch 23. In other embodiments, the snap-fit assemblies are optional and the electrodes may include alternate commercially available electrode components or a combination thereof. After the sensing device is attached to the adhesive patch 23, the patch is adhered to the maternal abdomen. The sensing device is designed to utilize the acoustic signals to identify and provide recommended placement locations to the user prior to placing the adhesive patch 23 to the maternal abdomen.

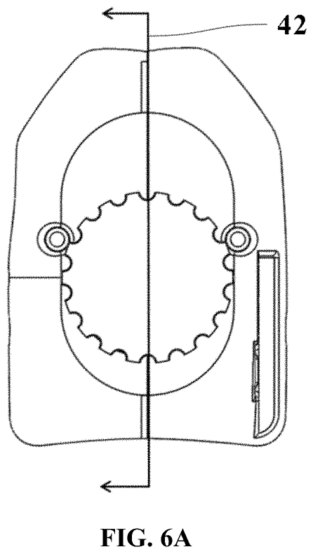

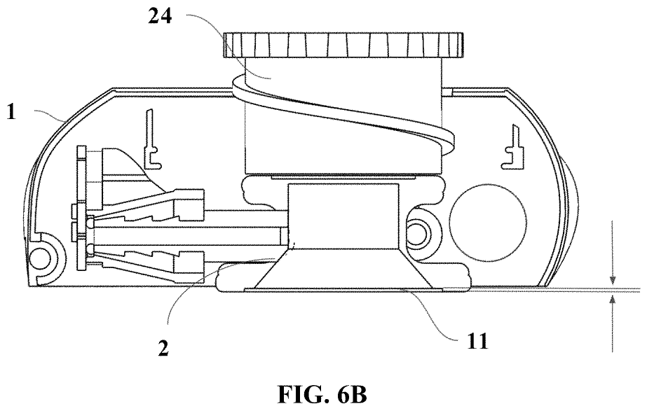

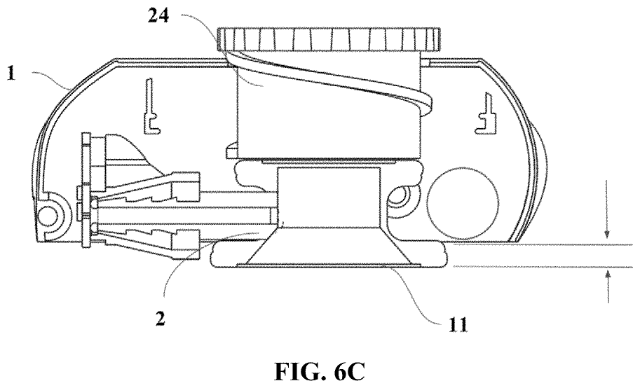

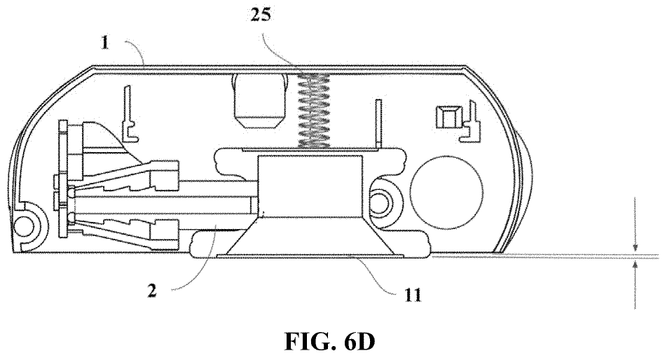

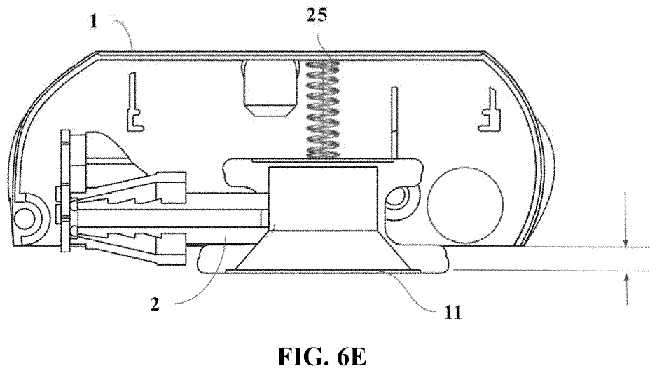

In an alternative embodiment, the pressure of the exposed frequency diaphragm 11 may be adjusted to improve signal quality. This pressure modification derives from the pressure modification component that extends the frequency diaphragm 11 relative to the housing 1 to achieve variable frequency diaphragm 11 pressures. In some embodiments, the pressure modification component is a screw 24, a spring 25, or a flexible garment 16. However, additional embodiments may include other pressure modification components designed to allow for adjustments of the pressure level(s) and to be fastened or otherwise secured during various levels of activity and movement including, but not limited to ambulation and sleep. In one embodiment, the pressure modification component is a screw 24 that is attached to the acoustic waveguide housing 3. Turning the screw 24 will extend or retract the frequency diaphragm 11. FIG. 6B shows an embodiment of the sensing device with a screw pressure modification component where the frequency diaphragm 11 in a retracted state. FIG. 6C shows an embodiment of the sensing device with a screw pressure modification component where the frequency diaphragm 11 in an extended state.

In an alternative embodiment, the pressure modification component is a spring 25 that is attached to the acoustic waveguide housing 3. The acoustic waveguide housing 3 moves along a slide fit assembly with designated pressure levels. FIG. 6D shows an embodiment of the sensing device with a spring pressure modification component where the frequency diaphragm 11 in a retracted state. FIG. 6E shows an embodiment of the sensing device with a spring pressure modification component where the frequency diaphragm 11 in an extended state.

As depicted in FIGS. 7A and 7B, the pressure modification component can be configured to display a visual pressure level indicator 26 to show the user the extension setting or distance of the frequency diaphragm 11 relative to the housing 1 of the device.

In an alternative embodiment, the attachment component and pressure modification component may be combined to create combinations of the embodiments that are configured to accommodate the needs of the user.

In an alternative embodiment, multiple sensing devices and/or acoustic peripherals can be placed on the maternal abdomen, or on other areas of the body, to increase the sensing area. An example acoustic peripheral includes: a sensing microphone 5, a noise-canceling microphone 6, and an acoustic waveguide 2. In one embodiment, the acoustic peripheral, or plurality of acoustic peripherals, can be connected and communicates with an MCU 8 on the main control PCB 10. In some embodiments an acoustic peripheral can communicate with the MCU 8 using pulse density modulation.

Fetal Heart Rate and Fetal Hemodynamics Signal Processing:

FIG. 8 shows an embodiment including the process of calculating FHR from the acoustic signal output of the sensing microphone 5 by the MCU 8. The signals shown in FIG. 8 have been normalized in the y-axis to +1, −1. In one embodiment, the technique can include the following steps: (1) The raw acoustic signal output 27 of the sensing microphone 5 is processed by the MCU 8. (2) The signal is filtered with a narrowband filter passing fetal heart frequencies 28. In alternate embodiments, the signal is not filtered, although improved signal quality can be achieved with signal filtering. (3) The amplitude of the acoustic signal is calculated using a sliding window of approximately 100 ms. The result is a signal 29 free of high frequency acoustic signals that increases in amplitude when the acoustic signal from a heartbeat is sensed. (4) A threshold is established to identify heart beats from the acoustic amplitude signal 30. (5) The time between identified beats (T_{b}) is calculated 31. (6) The average T_{b} for all identified beats is calculated (Mean-T_{b}). A FHR in beats per minute (BPM) 33 then calculated as FHR=(1/Mean-T_{b})*60.

In one embodiment, fetal hemodynamics can be calculated from the acoustic signal output of a sensing microphone 5 by the MCU 8. FIG. 8 shows this process, where the signals are normalized in the y-axis to +1, −1. In one embodiment, this technique can include: (1) The raw acoustic signal output 27 of the sensing microphone 5 is processed by the MCU 8. (2) The signal is filtered with a narrowband filter passing fetal heart frequencies 28. In alternate embodiments, the signal is not filtered, although improved signal quality can be achieved with signal filtering. (3) The amplitude of the acoustic signal is calculated using a sliding window of approximately 100 ms. The result is a signal free of high frequency acoustic signals 29 that increases in amplitude when the acoustic signal from a heartbeat is sensed. (4) A threshold is established to identify heart beats from the acoustic amplitude signal 30. (5) The amplitude of identified beats (A_{b}) is calculated 32. The arbitrary units (a.u) for this measurement are derived from the output acoustic signal amplitude of the sensing microphone 5 and may only be used as a relative assessment for stroke volume. It should be noted that while the y-axis of FIG. 8 has been normalized to +1,−1, the non-normalized acoustic signal is used in the calculation of A_{b}. (6) The average Ab for all identified beats is calculated (Mean-A_{b}). (7) A fetal relative stroke volume (FRSV) is equivalent to Mean-A_{b} 34. (8) A fetal relative cardiac output (RCO) is calculated by multiplying FHR and FRSV.

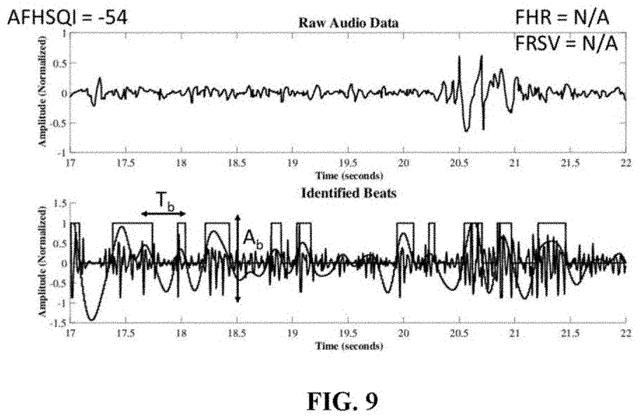

The AFHSQI can be calculated from the acoustic signal that is output from a sensing microphone 5 by a signal analysis system. FIG. 8 shows the AFHSQI in one embodiment for a high quality acoustic fetal heart signal detected at a recommended position and pressure. FIG. 9 shows the AFHSQI for an alternative embodiment with a low quality acoustic fetal heart signal detected with improper position and pressure. The following AFHSQI ranges can be used to define the qualitative signal quality:

AFHSQI>=110: excellent signal quality.

110>AFHSQI>=90: high signal quality.

90>AFHSQI>=70: acceptable signal quality.

70>AFHSQI>=50: poor signal quality.

AFHSQI<50: unacceptable signal quality. FHR and FRSV are not reliable.

The following method details the process for calculating AFHSQI according to one embodiment: (1) The raw acoustic signal output 27 of the sensing microphone 5 is processed by the signal analysis system. (2) The signal is filtered with a narrowband filter passing fetal heart frequencies 28. In alternate embodiments, the signal is not filtered, although improved signal quality can be achieved with signal filtering. (3) The amplitude of the acoustic signal can be calculated using a sliding window of approximately 100 ms. The result can be a signal free of high frequency acoustic signals 29 that increases in amplitude when the acoustic signal from a heartbeat is sensed. (4) A threshold is established to identify heart beats from the acoustic amplitude signal 30. (5) The time between identified beats (T_{b}) is calculated. (6) The standard deviation of T_{b} for all identified beats is calculated (STDT_{b}). (7) The amplitude of identified beats (A_{b}) is calculated 32. The arbitrary units (a.u) for this measurement are derived from the output acoustic signal amplitude of the microphone and may only be used as a relative assessment for stroke volume. It should be noted that while the y-axis of FIG. 8 has been normalized to +1,−1, the non-normalized acoustic signal is used in the calculation of A_{b}. (8) The average of A_{b} for all identified beats is calculated (MeanA_{b}). (9) The standard deviation of A_{b} for all identified beats is calculated (STDA_{b}). (10) T_{b}STD is then normalized to the expected high-quality measure of T_{b}STD (T_{b}-STD-NORM).

(a) Rate STD Quality Index (RSQI)=(T_{b}STDNORM−T_{b}STD)/T_{b}STDNORM

(11) A_{b}-STD is then normalized to the expected high-quality measure of A_{b}-STD (A_{b}-STD-NORM).

(a) Amplitude STD Quality Index (ASQI)=(A_{b}STDNORM−A_{b}STD)/A_{b}STDNORM

(12) A_{b}-Mean is then normalized to the expected high-quality measure of A_{b}-Mean (A_{b}-Mean-NORM).

(a) Amplitude Mean Quality Index (AMQI)=(A_{b}Mean−A_{b}MeanNORM)/A_{b}MeanNORM.

(13) The RSQI, ASQI, and AMQI are then averaged, multiplied by 100, and increased by 100, establishing the AFHSQI.

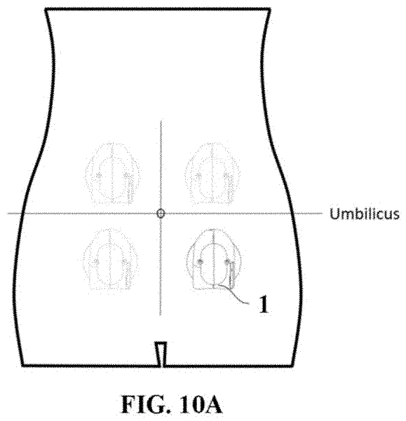

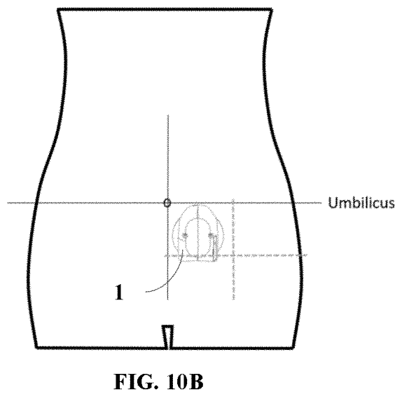

The AFHSQI can be leveraged in a subsequent method for determining the recommended position for measuring acoustic fetal activity. In one embodiment, as shown in FIG. 10A, the process involves dividing the maternal abdomen into quadrants. The quadrant with the highest calculated AFHSQI is then further subdivided into sub-quadrants. The sub-quadrants with the highest calculated AFHSQI are selected as the recommended position for measuring acoustic fetal activity. In alternate embodiments, a heart signal can be detected from alternate body parts, rather than the maternal abdomen. In some embodiments, the division can be alternate configurations including unequal or inconsistent sectioning. Sound produced from the fetal heart is omnidirectional making the acoustic measurement less sensitive to accurate placement position, which enables continuous operation. The fetal heart sounds measured by the sensing device are produced by the mechanical operation of the heart valves wherein the fetal heart sounds can be associated with hemodynamics. Detecting a recommended position and pressure level of the sensing device on the maternal abdomen can be relevant because maternal BMI and fetal position inside the womb will vary.

Returning the embodiment shown in FIG. 10A, the maternal abdomen can be subdivided into four equal quadrants with two perpendicular lines intersecting at the umbilicus of the maternal abdomen. The user or operator can then be instructed to position the sensing device in each of the quadrants, beginning with the first position and moving to a plurality of positions. Each time the user or operator confirms the position, the AFHSQI for the quadrant is calculated and stored in connection with the position, these stored calculations can be referred to as Q1-AFHSQI, Q2-AFHSQI, Q3-AFHSQI, Q4-AFHSQI in some embodiments, according to the corresponding quadrant. The recommended locations for sensor placement during this calibration stage and the instructions for placing and confirming the position can be provided through a user interface designed to receive a user or operator input.

Selecting the highest AFSQI from the Q1-AFHSQI, Q2-AFHSQI, Q3-AFHSQI, Q4-AFHSQI, the identified quadrant is further subdivided into 4 equal sized sub-quadrants as shown in FIG. 10B. The process is repeated and the sub-quadrant with the highest AFHSQI is identified. The process of quadrant subdivision may be repeated additional times, but in practice it is generally not required to subdivide the initial quadrants more than once. The identified sub-quadrant with the highest AFHSQI is the recommended position for measuring acoustic fetal heart activity and the recommendation is displayed on the user interface with instruction on the recommended location.

In a similar embodiment, the user can be instructed to adjust the pressure level between the sensing device and the maternal abdomen. The instructions for adjusting and confirming the pressure level can be provided through a user interface designed to receive a user or operator input. At each pressure level, the AFHSQI is measured. The pressure level with the highest AFHSQI is selected as the recommended pressure level for measuring acoustic fetal heart activity.

In one embodiment, the sensing device allows for adjustable pressure levels between the exposed frequency diaphragm 11, a face of the acoustic waveguide 2, and the maternal abdomen. There are at least two methods for accomplishing this using either an “attachment component” and a “pressure modification component”. In one embodiment the attachment component is the garment 16. When using the garment 16, the user may increase or decrease tension in the garment 16 to achieve differing pressure levels. In an embodiment using a pressure modification component, the frequency diaphragm 11 of the acoustic waveguide 2 can be configured to extend or retract relative to the housing 1 of the sensing device. In some embodiments the pressure modification component can be a spring, a screw, or similar device.

While any number of levels may be possible, in practice 4-5 levels are sufficient in most embodiments. At each pressure level, the user or operator confirms the pressure, and the AFHSQI is measured and recorded relative to the different quadrants the measurement is associated with, for example: L1-PR-AFHSQI, L2-PR-AFHSQI, L3-PR-AFHSQI up to LN-PR-AFHSQI. The pressure level with the highest AFHSQI (Recommended-Pressure-AFHSQI) is identified by the signal analysis system and the operator interface can instruct the operator to adjust the pressure to the recommended level. The signal analysis system may be configured to communicate alerts or other sensory indications to the user. In some embodiments, these alerts or indications may be associated with alarms, improper position or pressure, or failure to detect, recognize, or identify signals or calculated values described therein. Examples of sensory indication include, but are not limited to: visual, audio, haptics, or a combination thereof.

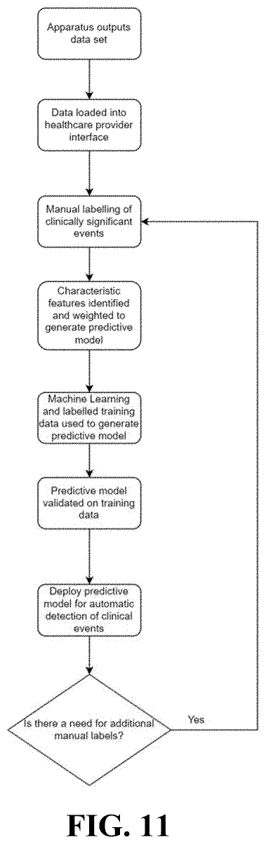

FIG. 11 shows an embodiment of the present invention related to a user interface that can be configured for a healthcare provider. The user interface can include a method and/or system for the healthcare provider to label clinically significant events and highlight related outputs of data correlated to those events. Additional embodiments include an interface and system for providing recommendation(s) about the detected clinically significant events that have been recognized and processed by the sensing device.

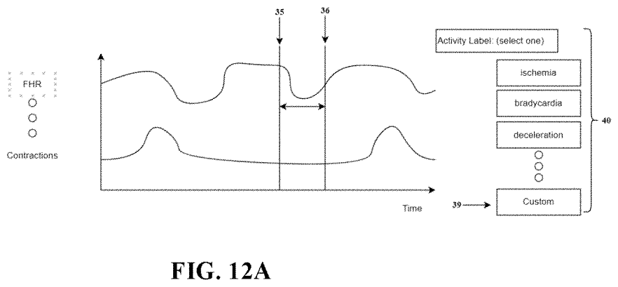

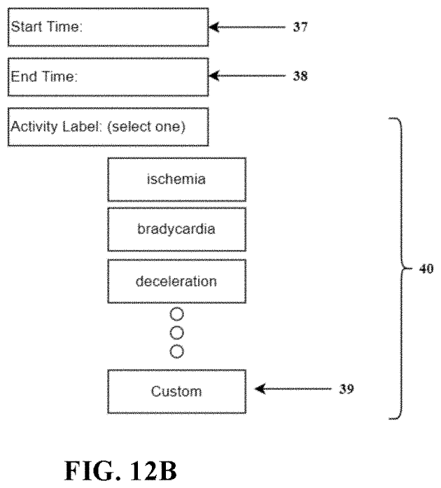

FIGS. 12A and 12B show an embodiment of the healthcare provider interface, configured to be used by a healthcare provider to input labels associated with clinically significant events. The labels may include data labels, activity labels, or other identifying information. Examples of clinically significant events may include, but are not limited to: clinically significant fetal heart activity, maternal uterine activity, and other activities related to labor. In one embodiment, the healthcare provider inputs a start time and end time where the output data of the apparatus indicates a clinically significant event and labels it from a provided set of options as shown in FIG. 12A. In another embodiment, the healthcare provider interface indicates start time and end time by moving cursors to the relevant position on the time axis in a graph of the output data as shown in FIG. 12B. The healthcare provider interface can include a method for labeling output data, the method comprising a first step including: (1) providing a visual display with data, wherein the data may include, but is not limited to: acoustic and biopotential signals associated with fetal heart activity, and maternal uterine activity. The healthcare provider interface can further include a start time and a stop time for the signal associated with a clinically significant event to be labeled. The method for labeling the data further comprises a second step including: (2) receiving an input from the healthcare provider and associating the input with either a start time, a stop time, or a combination thereof. In one embodiment, the input from the healthcare provider may include, for example, an input from a keyboard or by moving a cursor or a plurality of cursors on a visualization of the output data 35 and 36. In another embodiment, the healthcare provider can label the data by manually entering the start and stop time in a text box 37 and 38. The method for labeling the data can further include a third step, (3) providing a pre-populated label for the clinically significant event for the healthcare provider to select, or providing an option in a drop down menu 40 for the healthcare provider to add a custom label 39. The method for labeling data also includes a fourth step, (4) prompting the healthcare provider to review the label and confirm the inputs are correct. The label options could include, for example: ischemia, bradycardia, significant decelerations, and additional clinically significant events. Some embodiments include the option to configure a custom label. A training data set can be produced comprising the related labels, time stamps, and related data. A portion of the training data set could be used for validation and to provide recommendations to the healthcare provider. The healthcare provider interface can continue to permit the user, or healthcare provider to add additional manual labels to better enhance the recommendation feature.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A device for sensing perinatal maternal uterine activity and fetal heart activity, the device comprising:
a housing with an electromechanical system disposed therein;
a passive acoustic system, the passive acoustic system including a plurality of microphones and an acoustic waveguide;
an attachment component configured to secure the device securely against a human body;
an accelerometer for sensing motion activity;
a signal analysis system comprising a microcontroller unit configured to process acoustic and biopotential signals, wherein the microcontroller unit is configured to reduce motion artifacts from the acoustic and biopotential signals using the accelerometer; and
a wireless transceiver configured to share information between the signal analysis system and a base station with a user interface.

2. The device of claim 1, wherein the housing is provided in the form of a body that is acoustically isolated from the housing and the electromechanical system.

3. The device of claim 1, wherein the plurality of microphones include at least a sensing microphone and a noise-canceling microphone.

4. The device of claim 3, wherein the signal analysis system is further configured to remove external noise by subtracting the signal provided from the noise-canceling microphone from the signal provided from the sensing microphone.

5. The device of claim 1, wherein the passive acoustic system further comprises:
a resonance chamber; and
a tunable frequency diaphragm configured to transduce vibrations from the body into pressure waves.

6. The device of claim 1, wherein the passive acoustic system is configured to transduce a plurality of pressure waves, and the plurality of pressure waves are received by a sensing microphone.

7. The device of claim 1, wherein the acoustic waveguide includes a sealing component, the sealing component configured to maintain a sealed pressure path from a frequency diaphragm to a sensing microphone.

8. The device of claim 1, further comprising a pressure modification component is designed to improve signal quality by adjusting a pressure of a face of the acoustic waveguide against the body.

9. The device of claim 1, further comprising a plurality of electrodes in electrical communication with the signal analysis system.

10. The device of claim 9, wherein the attachment component is configured to integrate with the plurality of electrodes such that the attachment component provides an interface for external placement of the plurality of electrodes.

11. The device of claim 1, wherein the signal analysis system is configured to detect a fetal heart rate.

12. The device of claim 1, wherein the signal analysis system is configured to detect hemodynamics.

13. The device of claim 1, the signal analysis system further comprising: a main control printed circuit having the microcontroller unit, at least one microphone printed circuit board, a split power plane, and a ground plane, wherein the split power plane and the ground plane are positioned between the main control printed circuit board and the at least one microphone printed circuit board.

14. A method for determining the signal quality index of acoustic fetal heart sounds, the method comprising:
providing an electromechanical system for measuring fetal heart activity using a passive acoustic system;
processing the fetal heart activity using a signal analysis system;
generating an acoustic fetal heart signal from an output of the signal analysis system;
displaying instructions on a user interface for adjusting a position of the passive acoustic system and for adjusting a pressure of the passive acoustic system;
processing the fetal heart activity using the signal analysis system and recording the acoustic fetal heart signal in connection with the position of the passive acoustic system;
processing the fetal heart activity using the signal analysis system and recording the acoustic fetal heart signal in connection with the position of the passive acoustic system;
calculating an acoustic fetal heart signal quality index according to the output of the signal analysis system; and
updating the instructions on the user interface to include a recommended position and a recommended pressure of the passive acoustic system.

15. The method of claim 14, further comprising:
determining an acoustic fetal heart signal quality index with the signal analysis system by extracting a plurality of metrics from the acoustic fetal heart signal and aggregating the plurality of metrics into a signal quality index; and
displaying the acoustic fetal heart signal quality index on the user interface.

16. The method in claim 14, further comprising:
displaying instructions on the user interface for adjusting the position of the passive acoustic system at a first position on a human body;
receiving a user input, wherein the user input is a confirmation of the first position on the human body;
recording the fetal heart activity from the passive acoustic system;
calculating the acoustic fetal heart signal quality index;
storing a first acoustic fetal heart signal measurement associated with the acoustic fetal heart signal quality index at the first position;
displaying instructions on the user interface for adjusting a position of the passive acoustic system into a plurality of positions;
receiving the user input, wherein the user input is the confirmation of the plurality of positions on the human body;
recording, calculating, and storing the acoustic fetal heart signal measurement and the acoustic fetal heart signal quality index associated with each position of the plurality of positions;
comparing a stored value of the acoustic fetal heart signal index associated with each position of the plurality of positions;
selecting a highest acoustic fetal heart signal quality index from the stored value using the signal analysis system; and
displaying instructions on the user interface for adjusting a position of the passive acoustic system at the position associated with the highest acoustic fetal heart signal quality index.

17. The method in claim 14, further comprising:
displaying instructions on the user interface for adjusting a pressure level of the passive acoustic system, wherein the instructions can include defined pressure levels;
determining the acoustic fetal heart signal at each pressure level using the passive acoustic system;
outputting the acoustic fetal heart signal from the passive acoustic system to the signal analysis system;
processing the acoustic fetal heart signal using the signal analysis system;
measuring an acoustic fetal heart signal quality index at each pressure level;
comparing the stored acoustic fetal heart signal measurements;
selecting a highest acoustic fetal heart signal quality index from the stored values using the signal analysis system; and
displaying instructions on the user interface to adjust the pressure of the passive acoustic system at the pressure level associated with the highest acoustic fetal heart signal quality index.

18. A method for labeling a clinically significant event during labor with a user interface, the method comprising:
providing a visual display with a plurality of data, wherein the plurality of data is associated with a fetal heart activity and a maternal uterine activity;
receiving an input from a user;
associating the input with either a start time, a stop time, or a combination thereof;
providing a pre-populated label for the clinically significant event for the user to select; and
prompting the user to review a selected label and confirm the input or a plurality of inputs are correct.

19. The method of claim 18, wherein the plurality of data includes acoustic and biopotential signals associated with the clinically significant event.

20. The method of claim 18, further comprising: providing an option in a drop down menu for the user to add a custom label.
